(19) United States
(12) Patent Application Publication
(10) Pub. No.: US 2026/0244420 A1
(43) Pub. Date: Aug. 20, 2026

(54) DYNAMIC AI AGENT INSTANTIATION BASED ON AGENT FINGERPRINT

(71) Applicant: RPM-One, Inc. dba Drive Health, Gilbert, AZ (US)

(72) Inventors: Alexander SICULAR, Dallas, TX (US); Kevin LONGORIA, Gilbert, AZ (US); Rajkumar KHANDELWAL, Chandler, AZ (US)

(73) Assignee: RPM-One, Inc. dba Drive Health, Gilbert, AZ (US)

(21) Appl. No.: 19/417,730

(22) Filed: Dec. 12, 2025

Related U.S. Application Data
(60) Provisional application No. 63/760,607, filed on Feb. 19, 2025.

(51) Int. Cl. G06F 8/60; G06F 8/41; G06F 9/445; G06F 9/50; G06F 40/205

(52) U.S. Cl. CPC G06F 8/60; G06F 8/433; G06F 9/44505; G06F 9/5005; G06F 40/205

(57) ABSTRACT

Systems and methods for deterministic deployment of artificial intelligence agents are disclosed. A system can receive a deployment metadata file of an AI agent and a fingerprint for the AI agent, the deployment metadata file including identifiers of multiple components for deployment of an instance of the AI agent. The system can compute a hash of each component identified in the deployment metadata file and retrieved from storage. The system can assemble a second metadata file based on the hash of each component and a structure of the deployment metadata file. The system can validate the deployment metadata file based on verifying that a hash of the second metadata file matches the fingerprint for the AI agent. The system can, in response to the validation, provision the components identified in the deployment metadata file to deploy the instance of the AI agent.

### BACKGROUND

The present application claims the benefit of and priority to U.S. Provisional Application No. 63/760,607, filed Feb. 19, 2025, the disclosure of which is incorporated herein by reference in its entirety.

Artificial intelligence (AI) systems, including agentic AI systems, can include numerous interdependent software and data components that interact to perform computational tasks. Establishing consistency, authenticity, and integrity across these components can be difficult, particularly when systems are modified, updated, or deployed in distributed environments. For example, AI systems may be expected to achieve target performance criteria, but it can be computationally demanding to reliably determine that an AI system meets such criteria; the performance of the AI system can be non-deterministic, further limiting the ability to ensure that a given deployment of the AI system will meet the criteria.

### SUMMARY

AI systems, including agentic systems, can be deployed using any of a variety of components such as training data, model parameters, instruction sets, and software containers. These components can operate together to execute defined behaviors. Conventional deployment workflows for such agents can rely on manual configuration processes or version tracking systems that are unable to generate deterministic identifiers linking an agent to its constituent components. As a result, verification of the composition and provenance of an individual deployed agent can be unreliable, particularly across distributed systems where multiple copies or revisions of models and configuration files coexist. Traditional techniques for tracking agent configurations can use source control metadata or container image tags, which can lack cryptographic assurance that a particular deployed instance corresponds to a defined state. Consequently, control over lineage, reproducibility, and cross-system auditability of an AI agent can be subject to errors introduced by configuration drift, software variance, or unverified update processes. Any of various such technical errors can result in AI agent deployment that fails to meet appropriate performance criteria.

Systems and methods in accordance with the present disclosure can allow for deterministic and/or verifiable deployment of instances of AI agents. In some implementations, each component of the artificial intelligence agent, such as a data manifest, model definition, instruction set, and/or executable software package, can be processed by a hashing function to generate a corresponding component identifier. A metadata file can be assembled to include the component identifiers, manufacturer information, and dependency declarations that define the agent architecture. The metadata file can be canonicalized and hashed to produce the agent fingerprint, such that an immutable identity can be established for the underlying configuration. In some implementations, a separate fingerprint can be generated for a deployed instance by combining deployment-specific elements and environmental bindings with the base agent fingerprint, allowing deployment systems, remote systems, or verification authorities to perform cryptographic validation of an operating agent, including without requiring disclosure of proprietary components. The system can use the validation to more effectively provision components for deployment and execution of an instance of the AI agent.

Systems and methods in accordance with the present disclosure can allow for a deterministic process for generating a verifiable identifier for an AI agent, where the verifiable identifier can be used to trigger deployment of the AI agent. This can include creating an agent fingerprint derived from the component structure of the AI agent. In some implementations, the system can generate a metadata file that includes canonicalized component identifiers representing data, model, instructions, and/or software components associated with a manufacturer definition. The metadata file can be encrypted, such as to be hashed using a one-way cryptographic function, such that the resulting agent fingerprint provides a stable and reproducible reference derived directly from the agent's manifest. In some implementations, a deployed agent fingerprint can be generated by extending the agent fingerprint with user-specific components including customer data or configuration details. The combination of the manufacturer's fingerprint and deployment-specific information can produce a deterministic blueprint that can be used by orchestration systems for deployment and by credentialing entities for validation of provenance and integrity. For example, the system can implement a technical mechanism to uniquely identify an AI agent based on the agent fingerprint, and can define the methods by which various systems that deploy or otherwise rely on the AI agent can technically communicate and interact. This identity can serve as the foundation for an automated governance pipeline, such as where an agent can be automatically evaluated against the standards of a specific agentic credential, receive a formal license to operate within a given jurisdiction, and be insured based on its verifiable qualifications, for example and without limitation. Systems and methods as described herein can be applied for any of a variety of AI systems, including any of various training and/or inference pipelines, machine learning models, neural networks, language models, multimodal models, agentic systems, retrieval systems, reinforcement models, or combinations thereof.

At least one aspect relates to a system. The system can receive a deployment metadata file of an AI agent and a fingerprint for the AI agent. The deployment metadata file can include a plurality of identifiers corresponding to a plurality of components used for deployment of an instance of the AI agent. The system can compute a hash for each component identified in the deployment metadata file and retrieved from storage. The system can assemble a second metadata file based on the hash of each component and a structure of the deployment metadata file. The system can validate the deployment metadata file based on verification that a hash of the second metadata file matches the fingerprint for the AI agent. The system can, in response to validating the deployment metadata file, provision the plurality of components identified in the deployment metadata file to deploy the instance of the AI agent.

In some implementations, the system can trigger reception of the deployment metadata file based on detection of a change to the deployment metadata file within a repository. In some implementations, the system can parse the deployment metadata file to extract one or more hashes representing an identifier of at least one of a software component for the AI agent, a configuration for deployment of the AI agent, or a dependency indicating a networking policy for the AI agent. In some implementations, the system can deploy the AI agent based on the one or more hashes. In some implementations, the system can retrieve the plurality of components from a storage system provided by a manufacturer of the AI agent. In some implementations, the system can load instructions for deployment of the AI agent at runtime according to an identifier of the instructions in the plurality of identifiers. In some implementations, the fingerprint of the AI agent can represent a hash of a metadata file defining the AI agent. In some implementations, the system can output the deployment metadata file for credentialing the deployment of the AI agent. In some implementations, the plurality of components can include at least one of user-specific data for deployment of the AI agent or user-specific configuration information for operation of the AI agent. In some implementations, the plurality of identifiers can include at least one identifier for a hardware resource used to deploy the AI agent. In some implementations, the plurality of identifiers can indicate one or more dependencies required for deployment of the AI agent.

At least one other aspect relates to a method. The method can be performed, for example, by one or more processors coupled to non-transitory memory. The method can include receiving a deployment metadata file of an AI agent and a fingerprint for the AI agent, the deployment metadata file including multiple identifiers of components for deployment of an instance of the AI agent. The method can include computing a hash for each component identified in the deployment metadata file and retrieved from storage. The method can include assembling a second metadata file based on the hash of each component and a structure of the deployment metadata file. The method can include validating the deployment metadata file based on verification that a hash of the second metadata file matches the fingerprint for the AI agent. The method can include, in response to validation, provisioning the components identified in the deployment metadata file to deploy the instance of the AI agent.

In some implementations, the method can include triggering reception of the deployment metadata file based on detection of a change to the deployment metadata file in a repository. In some implementations, the method can include parsing the deployment metadata file to extract one or more hashes representing an identifier of a software component, a configuration, or a dependency indicating a networking policy for the AI agent, and deploying the AI agent based on the one or more hashes. In some implementations, the method can include retrieving the plurality of components from a storage system provided by a manufacturer of the AI agent. In some implementations, the method can include loading instructions for deployment of the AI agent at runtime based on an identifier of the instructions in the plurality of identifiers. In some implementations, the method can include validating the fingerprint of the AI agent as a hash of a metadata file defining the AI agent. In some implementations, the method can include outputting the deployment metadata file for credentialing the deployment of the AI agent. In some implementations, the method can include incorporating at least one of user-specific data or user-specific configuration information within the plurality of components. In some implementations, the method can include deploying the AI agent on one or more hardware resources identified by the plurality of identifiers. In some implementations, the method can include detecting dependencies for the deployment of the AI agent based on the plurality of identifiers.

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification. Aspects can be combined, and it will be readily appreciated that features described in the context of one aspect of the invention can be combined with other aspects. Aspects can be implemented in any convenient form, for example, by appropriate computer programs, which may be carried on appropriate carrier media (computer readable media), which may be tangible carrier media (e.g., disks) or intangible carrier media (e.g., communications signals). Aspects may also be implemented using any suitable apparatus, which may take the form of programmable computers running computer programs arranged to implement the aspect. As used in the specification and in the claims, the singular form of ‘a,’ ‘an,’ and ‘the’ include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is a block diagram of a system to generate an agent fingerprint, in accordance with one or more implementations;

FIG. 2 is a flow chart illustrating a method for generating an agent fingerprint, in accordance with one or more implementations;

FIG. 3 is a block diagram of an example of system to generate a deployed agent fingerprint, in accordance with one or more implementations;

FIG. 4 is a flowchart illustrating a method for generating and validating a deployment fingerprint for an AI agent, in accordance with one or more implementations;

FIG. 5 is a block diagram illustrating an example of a system to deploy AI agents, in accordance with one or more implementations; and

FIG. 6 is a flow chart illustrating a method for deploying an AI agent, in accordance with one or more implementations.

### DETAILED DESCRIPTION

Below are detailed descriptions of various concepts related to, and approaches, methods, apparatuses, and systems for implementing the various techniques described herein. The various concepts introduced above and discussed in greater detail below may be implemented in any of numerous ways, as the described concepts are not limited to any particular manner of implementation. Examples of specific implementations and applications are provided primarily for illustrative purposes.

The techniques described herein relate to systems and methods for deterministic deployment of AI agents. AI agents can be assembled from multiple interrelated components that may include data, trained models, software instructions, and external dependency definitions. Each component can contribute to the behavior and operation of an autonomous or semi-autonomous computing entity. The deployment of such agents can span cloud computing environments, edge devices, or virtualized compute infrastructures. In these environments, component composition can change frequently due to updates or reconfiguration of dependent elements. The ability to identify and describe each deployment through reproducible configuration data can support collaboration, auditability, and/or operational traceability.

Conventional approaches for deploying AI agents often rely on data that is descriptive but not cryptographically verifiable. Build systems or orchestrators can record version identifiers and configuration files, but such records can be mutable and may not establish a deterministic proof of the deployed artifact. When multiple instances of an agent share common containers or datasets, small divergences in versions or weakly tracked parameters can lead to configuration drift. This drift can cause inconsistencies between declared and operational states of the agent. Furthermore, the absence of strong linkage between deployment metadata and the underlying components can limit the ability of external parties to validate the integrity of a deployed agent without full disclosure of proprietary assets.

The techniques described herein can provide a verifiable framework in which an artificial intelligence agent can be associated with an immutable, cryptographically derived identifier referred to as an agent fingerprint. The agent fingerprint can represent a composite hash derived from individually hashed component manifests, such that the complete identity of an agent can be recovered from its constituent parts. A second derivation referred to as a deployed agent fingerprint can extend the base fingerprint by incorporating deployment-specific parameters, user-defined configurations, and environmental bindings. The two-tier structure can provide a hierarchical linkage between base models produced by a manufacturer and field-deployed instances operated by downstream systems (e.g., as managed by customers or partners). The system can use the agent fingerprint and/or the deployed agent fingerprint as both a cryptographically verifiable identifier for the AI agent and a blueprint or configuration that can be used to execute deployment of the AI agent.

In some examples, the techniques can generate the agent fingerprint by applying a cryptographic hash function to manifests that describe the agent's data sources, model definitions, instruction sets, and runtime software packages. The resulting component identifiers can be aggregated into a metadata file that defines the agent structure. The metadata file can then be canonicalized and hashed to compute a deterministic agent fingerprint. A similar process can be applied when producing a deployed agent fingerprint, where the system can incorporate customer data, deployment configurations, and dependency declarations. The same metadata files can also be used by deployment tools that instantiate the agent through automated workflows, thereby maintaining a cryptographically consistent link between the defined configuration and the physical execution environment. The system can use the agent fingerprint to trigger a configuration-driven deployment approach, which can ensure that any change to an agent's metadata file directly defines and automates its deployment process, enabling continuous integration and delivery with deterministic reproducibility across environments.

For example, the system can generate the agent fingerprint and/or deployed agent fingerprint to facilitate transparency and/or provenance, such as to provide an immutable bill of materials for any given AI agent. The system can provide a technical solution for trust and/or confidence in the AI agent, such as to enable rigorous and/or independent credentialing and licensing. The system can facilitate traceability and/or auditability, as each action executed by a deployed agent can be traced back to the deployed agent fingerprint, which can serve as the unique, verifiable identity for the deployed agent, which can create a clear audit trail. The system can facilitate accountability and/or liability; for example, by uniquely identifying an agent (as well as the provenance of its underlying components), liability for its actions can be more clearly assessed and risk adjusted, which can facilitate a technical implementation for a functional insurance market. The system can provide for greater reproducibility, as the fingerprinting mechanism can ensure that a certified agent's behavior can be precisely reproduced for testing, validation, and/or incident investigation. The system can allow for technical implementation of safety and/or recall functionality across deployments of AI agents, as the ability to uniquely identify every agent instance in the field can allow for targeted recalls or deactivations if a flaw or vulnerability is discovered.

A computing system implementing the techniques described herein can include one or more processors configured to hash and assemble component manifests into structured data objects, which can in turn be hashed to generate a machine-readable fingerprint for the AI agent. The system can create a metadata file containing component identifiers for data, model, instruction, and software elements. The metadata file can then be canonicalized and subjected to an encryption or hashing process, producing a deterministic fingerprint of the AI agent. For deployment, the system can receive additional user-defined components, generate new hashes for those components, and construct a deployment metadata file. Encryption of this deployment file can yield a deployment fingerprint that references the base agent fingerprint (e.g., to provide a provenance link to the AI agent as provided by the manufacturer), providing a verifiable linkage between the manufacturer's configuration and the deployed implementation. The resulting metadata structures can be stored in repositories (e.g., stored in artifact registries and tagged on container images) and used as configuration sources for automated orchestration or compliance evidence. For example, the metadata structures can be used to valid a candidate configuration of an AI agent against a reference configuration (e.g., as indicated by the agent fingerprint), which, upon validation, can be used as a blueprint to trigger configuration, orchestration, and/or deployment of the AI agent according to the metadata structure, such as to provision components of the AI agent according to the identifiers of the components in the metadata structure, along with any dependencies that are in the metadata structure.

The techniques described herein can provide a reliable method for maintaining integrity, traceability, and/or reproducibility of AI agents throughout their lifecycle. By deriving fingerprints directly from canonicalized component data, the techniques eliminate inconsistencies introduced by environmental or human factors. Validation entities can verify that a running agent matches its declared configuration by recomputing the fingerprints from observed components. Deployment orchestrators can use the same metadata to instantiate consistent environments without manual intervention. Through these technical capabilities, the approaches described herein can establish a deterministic link between identity, configuration, and execution for AI agents.

The techniques described herein can improve the reliability and verifiability of distributed AI deployments. By anchoring each deployment to a deterministic fingerprint, the disclosed methods can reduce ambiguity caused by configuration drift and mutable versioning information. The mechanisms can provide a consistent proof of identity across independent verification systems such that credentialing, licensing, or auditing processes can validate an operating agent without direct access to its internal components. These techniques can thereby strengthen data provenance, reproducibility, and accountability in the deployment of artificial intelligence agents across cloud, edge, and on-premises environments.

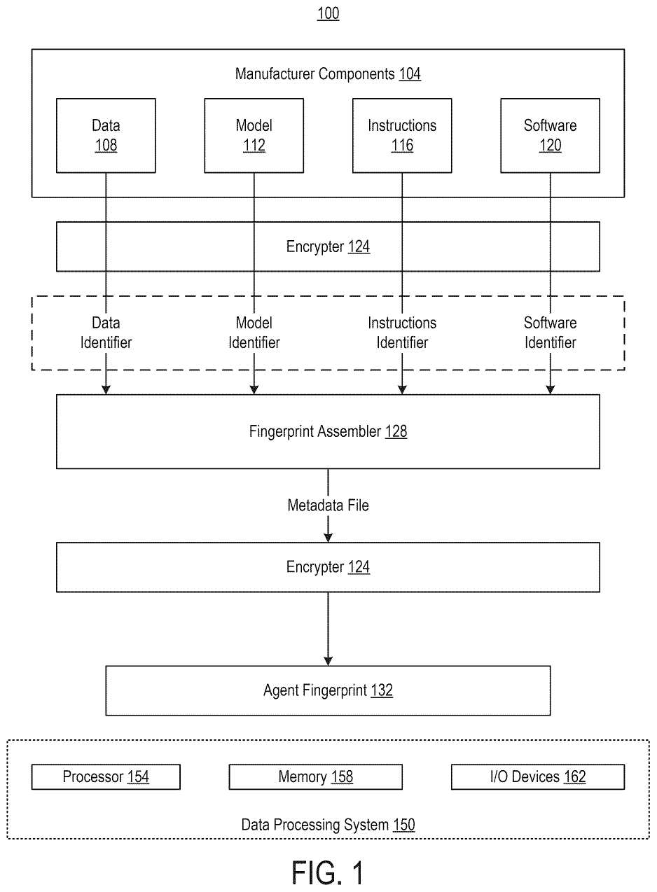

Referring now to FIG. 1, in brief overview, illustrated is a block diagram of a system 100, such as an agent fingerprint generation system 100. The system 100 can generate a deterministic identifier of an AI agent, derived from manufacturer components of the AI agent. For example, the system 100 can include manufacturer components 104, which can include data 108, model 112, instructions 116, and software 120. The system 100 can further include an encrypter 124 and a fingerprint assembler 128, and can output an agent fingerprint 132. The system 100 can include or be implemented using one or more data processing systems 150.

Referring to FIG. 1 in further detail, the system 100 can be or include a computing platform (e.g., data processing system 150) that processes component data of an AI agent to produce a deterministic fingerprint representing the AI agent. For example, the system 100 can be implemented as at least one of a cloud-based build pipeline or an on-premises system including one or more CPUs or GPUs. The system 100 can obtain component manifests, can generate hashes, and can assemble these into metadata files used to define an agent's identity. As an example, the system 100 can compute cryptographic digests for datasets, models, instruction files, and container images, which the system 100 can combine into a canonical metadata structure. The system 100 can perform deterministic hashing and metadata assembly procedures to compute a reproducible agent fingerprint based on canonical ordering rules. For example, the system 100 can use encryption, e.g., SHA-256 encryption, and can apply JSON canonicalization, to avoid environmental variance across builds. As depicted in FIG. 1, the data processing system 150 can include one or more processors 154, one or more memory (e.g., memory devices) 158, and/or one or more input/output (I/O) devices 162. The processor 154 can be a general purpose or specific purpose processor, an application specific integrated circuit (ASIC), one or more field programmable gate arrays (FPGAs), a group of processing components, or other suitable processing components. The processor 154 may be configured to execute computer code or instructions stored in memory (e.g., fuzzy logic, etc.) or received from other computer readable media (e.g., CDROM, network storage, a remote server, etc.) to perform one or more of the processes described herein. The memory 158 may include one or more data storage devices (e.g., memory units, memory devices, computer-readable storage media, etc.) configured to store data, computer code, executable instructions, or other forms of computer-readable information. The memory 158 may include random access memory (RAM), read-only memory (ROM), hard drive storage, temporary storage, non-volatile memory, flash memory, optical memory, or any other suitable memory for storing software objects and/or computer instructions. The processor 154 can be implemented as a hardware processor including a Central Processing Unit (CPU), an Application-Specific Integrated Circuit (ASIC), an Application-Specific Instruction-Set Processor (ASIP), a Graphics Processing Unit (GPU), a Physics Processing Unit (PPU), a Digital Signal Processor (DSP), a Field Programmable Gate Array (FPGA), a Programmable Logic Device (PLD), a Controller, a Microcontroller unit, a Processor, a Microprocessor, an ARM, or the like, or any combination thereof. The memory 158 may include database components, object code components, script components, or any other type of information structure for supporting the various activities and information structures described in the present disclosure. The memory 158 can include various modules (e.g., circuits, engines) for completing processes described herein. The I/O devices 162 can include any one or more communications electronics (e.g., wired or wireless reception and/or transmission circuitry; communications busses; etc.), and can include any one or more user interface devices (e.g., displays, microphones, keyboards, mouse devices, touch input devices, etc.) to facilitate communication with the data processing system 150 and/or one or more components thereof. The data processing system 150 can be implemented in any of various computing platforms or architectures, including but not limited to any of various client-server architectures.

As illustrated in FIG. 1, the system 100 can include or obtain one or more components 104, such as manufacturer components 104 that a manufacturer of an AI agent provides to facilitate execution or deployment of the AI agent. The components 104 can be or include any one or more data structures, software, firmware, code, scripts, or pointers or identifiers thereof. The manufacturer can include an entity that develops, stores, or provides the components 104, e.g., via one or more repositories. For example, the manufacturer can be a manufacturer of record of the AI agent. The AI agent can include or be defined according to one or more of the components 104.

The AI agent can include one or more neural networks, language models, multimodal models, or combinations thereof, such as represented by model 112 of the components 104. The AI agent can be a self-contained computational entity that performs one or more tasks autonomously by processing data and executing algorithms that emulate human reasoning, decision-making, or problem-solving, for example and without limitation. The AI agent can include multiple interdependent components such as a trained model (e.g., trained model 112), data inputs (e.g., data 108), instructions (e.g., prompts and/or instructions 116), and/or executable software (e.g., software 120), which can determine or manage the operation or behavior of the AI agent. In some implementations, the AI agent can operate as a composite system integrating neural network architectures, reinforcement learning modules, and rule-based logic to generate context-dependent outputs. For example, a language-processing AI agent can receive textual inputs, can tokenize the inputs into interpretable elements, and can produce coherent responses derived from a parameterized transformer model. In some implementations, the AI agent can interact with external systems and data stores, can apply learned policies to dynamic environments, and can (continuously) update its internal state variables to optimize performance metrics. The AI agent can process numeric, categorical, linguistic, or multimodal data and can execute within a distributed infrastructure that includes cloud-based servers, local computing nodes, or edge devices. Each AI agent can maintain an internal configuration defining the parameters, data references, and operational contexts under which it performs assigned tasks, allowing deterministic instantiations for analysis, testing, and deployment across technical environments.

Referring further to FIG. 1, the components 104 can be elements (e.g., provided by the manufacturer) of a base definition of the AI agent. The base definition can represent, for example, a minimum or sufficient set of components 104 to allow for functionality of the AI agent (e.g., even if additional user data or other resources may be expected to be used to facilitate deployment of the AI agent according to one or more criteria of a user). For example, the components 104 may represent data files, trained models, instruction sets, and/or software containers, such as may be generated under controlled build conditions. The manufacturer components 104 can serve as the inputs from which individual component identifiers are computed for inclusion in the agent fingerprint, as described further herein.

The components 104 can include data 108. The data 108 can include any of various datasets, data manifests, or data assets that the AI agent (e.g., model 112) uses for training (including any of various unsupervised learning, supervised learning, fine-tuning, transfer learning, or in context-learning) and/or inference (including, for example and without limitation, for context data that the model 112 refers to in order to perform inference, or for retrieval, such as for retrieval-augmented generation (RAG)). For example, the data 108 may include structured medical datasets, language corpora, or tabular data used for specific domain adaptation. The data 108 can include any of various text, speech, audio, image, and/or video data. The data 108 can include structured or unstructured data. The data 108 can include training data pairs, such as training data elements that are each associated with respective labels.

The components 104 can include at least one model 112. The model 112 can include any one or more functions, algorithms, machine learning models, neural networks, reinforcement learning models, language models, large language models (LLMs), small language models (SLMs), multimodal language models, or combinations thereof. The model 112 can include data representing the structure of the model 112, such as any one or more configurations of the model 112, such as weights, biases, parameters, versions, base models, architectures, tuning parameters, arrangements or types of network layers, connections between layers, types of input data or output heads, or various combinations thereof. For example, the model 112 can represent a trained neural network or statistical model implementing an inferencing capability of the AI agent. For example, the model 112 may correspond to a large language model, a fine-tuned transformer, or other parametric architecture.

In some implementations, the components 104 include instructions 116. The instructions 116 can include at least one of instructions or prompts that the model 112 can process to perform corresponding actions. In some implementations, the model 112 uses at least a portion of instructions 116 as context. In some implementations, the model 112 can combine (e.g., append) one or more instructions 116 to prompts received from a user or other system to input to the model 112, e.g., to input to the AI agent The instructions 116 can represent operational control data or prompt templates directing behavior of the AI agent. For example, instructions 116 may be files including initialization prompts or system configurations guiding contextual responses.

In some implementations, the components 104 can include software 120. The software 120 can include or be coupled with or reference any one or more code, scripts or firmware, for example, that can execute the AI agent, such as to provide at least one of an application layer or an interface for or to the AI agent. For example, the software 120 can include executable code or a containerized image that packages runtime dependencies for the AI agent. For example, the software 120 can include container image digests that encapsulate environment variables, dependency libraries, and operating system layer hashes.

The agent fingerprint generation system 100 can include an encrypter 124. The encrypter 124 can include any one or more code, scripts, software, algorithms, functions, rules, or combinations thereof to perform operations such as applying an encryption, such as a cryptographic operation, on components 104. For example, the encrypter 124 can be a cryptographic engine, and can computes one or more hashes (e.g., hash values) of data inputted to the encrypter 124, such as to compute hashes of the respective components 104. In some implementations, the encrypter 124 includes a tokenizer or an embedding model. In some implementations, the encrypter 124 can implement a Federal Information Protection Standard (FIPS)-compliant algorithm to perform the encryption. The encrypter 124 can apply a cryptographic operation, such as SHA-256 or another deterministic hash function, to the components 104. For example, as described further herein, the encrypter 124 can receive a component 104 (e.g., any of the data 108, model 112, instructions 116, and/or software 120), and compute a hash (e.g., a cryptographic hash) of the component 104 to generate a corresponding identifier of the component (e.g., data identifier of the data 108; model identifier of the model 112; instructions identifier of the instructions 116, software identifier of the software 120). The encrypter 124 can generate the hashes to be encoded or encrypted representations of respective components 104, such as machine-readable representations.

In some implementations, the encrypter 124 hashes each of the components 104 to produce the identifiers as unique identifiers (which collectively can form the metadata record and/or metadata file). The encrypter 124 can ingest the components 104 for cryptographic derivation of the identifiers. As an example, the encrypter 124 can pass each file or manifest of the components 104 into an SHA-256 hashing process, to generate the respective identifier.

For example, the encrypter 124 can hash the data 108 to generate the data identifier. The data identifier can be a digest that represents a canonical form of the data 108. For example, the encrypter 124 may receive a manifest describing source repositories or data revisions, and can output a single cryptographic hash as the data identifier. The data identifier can be used by a deployment system to mount a correct, versioned dataset for the AI agent.

The encrypter 124 can hash the model 112 to generate the model identifier. The model identifier can convey the version and parameter configuration used in the build of the model 112. For example, the encrypter 124 can hash the model 112 to include the model architecture file and the configuration parameters of model 112 in a manifest represented by the model identifier, such as where a checksum of the manifest forms the model identifier. The model 112 may be input to the encrypter 124, which can compute the model identifier as a cryptographic hash of the model 112. The model identifier can be used by a deployment system to load the correct model files.

The encrypter 124 can hash the instructions 116 to generate the instructions identifier. The instructions identifier can be a unique identifier describing the instructions 116, such as where the instructions 116 include a version-controlled instruction set. For instance, the instructions identifier can be mapped to a version-controlled file (e.g., via Git commit hash) that is loaded by a deployment system at runtime.

The encrypter 124 can hash the software 120 to generate the software identifier. The software identifier can specify an exact executable environment for deployment of the AI agent. For example, the software identifier represent a specific and/or pullable container image digest (e.g., sha256: . . . ) that an orchestrator, such as Kubernetes, can used to deploy the exact software build for the AI agent. For example, the software identifier can be used as a direct and/or executable pointer.

Referring further to FIG. 1, the system 100 can include a fingerprint assembler 128. The fingerprint assembler 128 can include any one or more functions, algorithms, rules, policies, heuristics, models, or combinations thereof to perform operations such as to generate a data structure, such as a metadata file, based at least on the identifiers of the components 104 that the encrypter 124 generates. For example, the fingerprint assembler 128 can generate the metadata file according to a structure for the metadata file, such as an order of inclusion of data in fields of the metadata file. For example and without limitation, the structure can represent a set of name-value pairs, ordered lists, and/or comma-separated values. The fingerprint assembler 128 can generate the metadata file as a JSON file.

In some implementations, the fingerprint assembler 128 generates the metadata file to include an identifier of the manufacturer of the AI agent. For example, the fingerprint assembler 128 can use the encrypter 124 to hash a name or other identifier of the manufacturer to generate the identifier of the manufacturer. The fingerprint assembler 128 can generate the metadata file to include an identifier of the AI agent, such as a name or version of the AI agent; the identifier of the AI agent may be human-readable (or can be hashed to be a machine-readable identifier).

The fingerprint assembler 128 can generate the metadata file to include the identifiers of the components 104 from the encrypter 124. For example, the fingerprint assembler 128 can assemble the data identifier, model identifier, instructions identifier, and software identifier into the structure of the metadata file. By referencing the identifiers, the metadata file can allow for reproducible access to the components 104 (e.g., referencing the data identifier can allow for reproducible access to the datasets of the data 108 to use for operation of the AI agent).

The fingerprint assembler 128 can combine the identifiers into the structure of the metadata file, which can be used for agent validation and future deployments. The fingerprint assembler 128 can generate the metadata file to include data for one or more fields of the identifier of the manufacturer, the identifier of the AI agent, a timestamp indicating when the AI agent build was finalized, the identifiers of the components 104, and/or external dependency manifest data. The fingerprint assembler 128 can perform canonical ordering (e.g., according to a metadata schema) of the name-value pairs of such fields to prepare the structure for cryptographic hashing. As an example, the fingerprint assembler 128 can perform key ordering and array normalization steps to avoid nondeterministic serialization. In some implementations, the fingerprint assembler 128 generates the metadata file to include a version string for the metadata schema, which can further facilitate reliable use of the metadata file.

In some implementations, the fingerprint assembler 128 generates the metadata file to include (one or more identifiers of) one or more manifests of external dependencies for the AI agent. This can include, for example, an array declaring the types of external services the AI agent is architected to use, acting as a manifest of approved tool slots. The manifest of external dependencies can be used by a deployment system to pre-configure network access or service bindings, for example.

In some implementations, the fingerprint assembler 128 generates the metadata file to include (one or more identifiers of) an interface specification for the AI agent. For example, the fingerprint assembler 128 can include an identifier, such as a hash, of the interface specification (e.g., to a remote system, such as a model provider) that the dependency is to conform to, such as for automated client generation or interface testing.

The following is an illustrative example of a metadata file, including the identifiers and the schema for the metadata file:

{

“format_version”: “1.1”,

“manufacturer_id”: “87b1c428-2c67-428a-a195-21a4c4202c2e”,

“agent_model_name”: “MediBot-Nurse-v3.2-Intake”,

“timestamp”: “2025-08-27T10:00:00Z”,

“components”: {

“data_cid”:

“sha256: a1b2c3d4e5f678901234567890abcdef1234567890abcdef1234567890ab”,

“model_cid”:

“sha256: b2c3d4e5f6a1234567890abcdef1234567890abcdef1234567890abcde”,

“instructions_cid”:

“sha256: c3d4e5f6a1b234567890abcdef1234567890abcdef1234567890abcd”,

“software_cid”: “sha256: d4e5f6a1b2c34567890abcdef1234567890abcdef1234567890abcde”

},

“external_dependencies_manifest”: [

{

“dependency_id”: “EMBEDDINGS_PROVIDER”,

“description”: “Service for generating text embeddings for RAG.”,

“interface_spec_cid”: “sha256: 2b3c4d5e6f7a8901234567890abcdef1234567890abcdef1234567890”

},

{

“dependency_id”: “PATIENT_LOOKUP_API”,

“description”: “Tool for retrieving patient records from an EMR.”,

“interface_spec_cid”: “sha256: 3c4d5e6f7a2b901234567890abcdef1234567890abcdef123456789”

}

]

}

Referring further to FIG. 1, the encrypter 124 (which can be a same encrypter 124 that encrypts the components 104 into respective data, model, instructions, and/or software identifiers, or a different encrypter 124 or instance of an encrypter 124) can encrypt the metadata file to generate an agent fingerprint 132. For example, the encrypter 124 can hash the metadata file (e.g. and without limitation, using SHA256) to generate the agent fingerprint 132. The agent fingerprint 132 can be a cryptographic representation of the metadata file, such as to allow for a compact and/or verifiable representation of the AI agent and the components 104 used to deploy the AI agent.

The agent fingerprint 132 can represent the unique, deterministic identifier of the AI agent as produced by the manufacturer. For example, the agent fingerprint 132 can be the SHA-256 digest of the canonicalized AF metadata file describing all core components. The agent fingerprint 132 can be used as a verifiable reference or blueprint to validate any instance of the AI agent during deployment or credentialing. The system 100 can store the agent fingerprint 132 may be stored in a registry or deployment system for retrieval during later validation or licensing workflows. As an example, the system 100 can apply the agent fingerprint as a metadata tag, e.g., an immutable metadata tag, to a container image for the AI agent. This can allow the system 100 to facilitate verifiable use of the AI agent upon retrieval of the container image.

Referring further to FIG. 1, the system 100, e.g., using the encrypter 124, can generate component identifiers from manufacturer component inputs, can assemble the component identifiers (along with any of various other identifiers as noted above) into the structure of the metadata file, and can hash the assembled metadata file to form the agent fingerprint 132. For instance, the system 100 can first compute per-component identifiers, and can subsequently execute a final hash pass over the canonicalized JSON object produced by the fingerprint assembler 128. The encrypter 124 may perform these operations by serializing component manifests into a canonical format before computing digest outputs. As an example, canonicalization may include alphabetically ordering keys and removing extraneous whitespace prior to the hash computation.

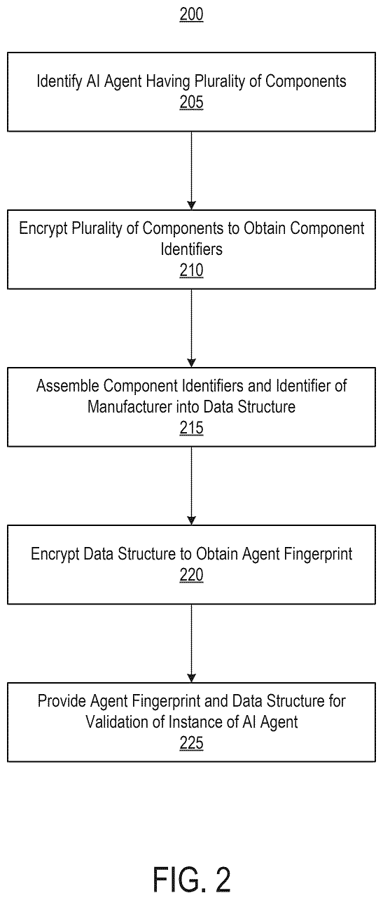

Referring now to FIG. 2, illustrated is a flow chart of a method 200 for generating an agent fingerprint for an AI agent. The method 200 can be executed, performed, or otherwise carried out by any of various systems described herein, including one or more components of the system 100. In brief overview of the method 200, the method 200 can include identifying an AI agent having a plurality of components 205, encrypting the plurality of components to obtain component identifiers 210, assembling the component identifiers and a manufacturer identifier into a data structure 215, encrypting the data structure to obtain an agent fingerprint 220, and providing the agent fingerprint and the data structure for validation of an instance of the AI agent 225. The method 200 or one or more operations of the method 200 can be triggered responsive to any of a variety of events, such as a request for validation or deployment of the AI agent, or in response to detection of a change in one or more components (e.g., components 104) of the AI agent.

At 205, the method 200 can include identifying an AI agent that includes or is associated with a plurality of components. For example, one or more manifests or repositories that define the constituent components of the AI agent can be accessed. Atomic elements for the AI agent such as data files, model parameters, instruction manifests, and/or executable software components can be identified, each representing a discrete element of the agent's operational configuration. In some implementations, version-controlled directories associated with a build environment of the AI agent can be processed to obtain resource descriptors that collectively define an operational scope for the agent. For example, the agent fingerprint generation system can enumerate structured references identifying model artifacts, data resources used for retrieval-augmented generation (RAG), container image references, and interface dependency manifests stored in respective repositories. The identification can occur at build initialization or when a manufacturer prepares a release candidate for fingerprint generation, such as during an automated continuous integration or continuous deployment pipeline that executes after source repositories containing model, data, and configuration definitions are checked out. The identification can occur responsive to a request to deploy or validate the AI agent.

At 210, the method 200 can include encrypting the plurality of components to obtain component identifiers of the plurality of components. Each component of the AI agent can be processed (e.g., encrypted, encoded) to generate a deterministic hash value, which can uniquely represent the data or content of the respective component content. In some implementations, a FIPS compliant cryptographic function such as Secure Hash Algorithm 256 (SHA-256) can be applied to each retrieved manifest for each component, which describes inputs including data, model, instructions, and/or software. For example, a base data manifest, a model configuration file, an instruction definition, and a container manifest can be read, serialized into a canonicalized form, and subjected to computation of corresponding cryptographic digests, thereby producing a set of component identifiers. In some implementations, canonicalization can involve ordering keys alphabetically, normalizing character encoding, and eliminating redundant whitespace to ensure that identical manifests always yield the same digest output. For example, a JSON metadata object representing a model configuration can be normalized prior to execution of the SHA-256 algorithm, which can produce a reproducible identifier value that accurately reflects the state of the model inputs within the manufacturing environment.

At 215, the method 200 can include assembling the component identifiers and an identifier of a manufacturer into a data structure, such as a data structure for a metadata file. For example, the identifiers of the components, an identifier of the manufacturer of the AI agent, and/or additional metadata such as timestamps or dependency manifests can be combined into a structured data object. A structured object can be generated as a metadata file containing name-value pairs that associate each identifier with a corresponding field defining the component type. In some implementations, identifiers of the data, model, instructions, and/or software for the AI agent can be merged into the metadata file. The assembly operation can be performed automatically after all component identifiers have been generated, thereby consolidating the component data into a complete and deterministic bill of materials. For example, in a continuous integration pipeline, the assembly can be triggered automatically at completion of the final component identifier hashing process to produce a finalized metadata object. The metadata file can be formatted in accordance with predefined canonicalization rules specifying serialization order and syntax consistency. In some implementations, alphabetical ordering of field names can be maintained, and consistent serialization can be applied across records to allow for reliable reproduction of identical cryptographic verification outputs across environments.

At 220, the method 200 can include encrypting the data structure (e.g., the metadata file) to obtain an agent fingerprint. For example, responsive to assembly of the metadata file, a cryptographic hash can be applied to the metadata file to generate the agent fingerprint, which can provide for a deterministic identifier of the AI agent and the components of the AI agent. In some implementations, the agent fingerprint can be expressed as AF=SHA-256 (Canonicalize(AF_Metadata_File)), resulting in a reproducible digital signature derived from the canonicalized metadata content. The hashing operation can be performed after the assembly process is finalized, such as prior to the storage or distribution of the generated fingerprint. In some implementations, hashing can be executed during a final artifact packaging sequence within a continuous integration environment before release to a registry. The output of the hash computation can be verified through checksum comparison to detect any divergence between the computed value and expected reference data.

The method 200 can include providing the agent fingerprint and the data structure (e.g., the metadata file) for validation of an instance of the AI agent (225). For example, the agent fingerprint and the corresponding metadata file can be transmitted or otherwise made accessible to validation, credentialing, or licensing entities for reference in subsequent verification processes. In some implementations, the generated data can be uploaded to a credential-granting organization or deposited within an attestation registry that permits access by authorized verification systems. For example, completion of fingerprint generation and archival operations can precede the transmission phase, allowing the information to be used as a reference record for deployment validation or credential assessment. In some implementations, distribution of the fingerprint and metadata can occur during the final build stage to align the release of the agent with the initiation of credential verification procedures. The fingerprint and metadata can be provided through one or more secure interfaces that facilitate retrieval and comparison of encrypted component identifiers. For example, a verification authority can load a corresponding deployed container image, regenerate an associated fingerprint, and confirm alignment with the manufacturer-declared fingerprint to validate authenticity of the agent instance.

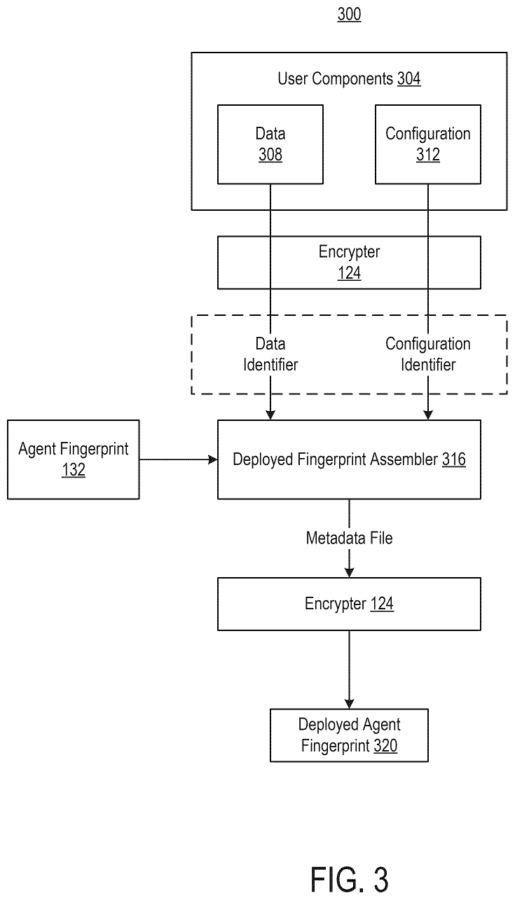

Referring now to FIG. 3, illustrated is a block diagram of a system 300, such as a system for generating a deployed agent fingerprint for a deployed instance of an AI agent. The AI agent can correspond to the AI agent described with reference to FIG. 1, where the deployed instance can be further updated (e.g., customized, modified, trained, fine-tuned, etc.) for a target application, such as for a customer and/or user of the AI agent (e.g., in contrast to the base definition of the AI agent that may be represented by the components 104 and/or agent fingerprint 132). The system 300 can be implemented to generate the deployed agent fingerprint based on customer-specific components and a previously established agent fingerprint. In some implementations, the system 300 obtains the agent fingerprint 132 that the system 100 generates, for example. In brief overview, the system 300 can include one or more user components 304, the encrypter 124, and a deployed fingerprint assembler 316, and can output a deployed agent fingerprint 320. The user components 304 can include data 308 and configuration 312.

As depicted in FIG. 3, the system 300 can include or obtain one or more components 304, which can be user components 304. For example, the components 304 can represent deployment-specific assets supplied by a user, customer, or end organization for use with the AI agent. For example, the user components 304 can include customer-provided data sources, configuration files, and local environment manifests utilized during deployment. The AI agent can use the components 304 to deploy a user-specific (e.g., customer-specific) instance of the AI agent.

The components 304 can include data 308. The data 308 can include one or more datasets or resource manifests for the AI agent. In some implementations, the data 308 includes domain-specific information and/or data for RAG operations that the AI agent is to perform. For example, the data 308 can include medical records datasets, financial policy tables, or other proprietary information local to the deploying organization. The data 308 can include data from or identifiers of any of various data sources.

The user components 304 can include at least one configuration 312. The configuration 312 can include one or more files or manifests that define parameters and/or settings for the deployment of the AI agent. For example, the configuration 312 can specify parameters such as environment variables, model bindings, or resource allocation limits unique to the target environment (e.g., software and/or hardware environment) in which the AI agent is to be deployed.

Referring further to FIG. 3, the system 300 can include the encrypter 124. The encrypter 124 can be configured for the system 300 in a manner analogous to the encrypter 124 of the system 100 (though may be implemented using one or more separate encrypters or encryption functions than used by the system 100). The encrypter 124 can encrypt the components 304 to generate identifiers of the components 304, such as to compute respective hashes of the data 308 and/or configuration 312 to generate a data identifier and/or a configuration identifier. As an example, each asset within the components 304 can be hashed to form identifiers which, as described further herein, the system 300 can append to a metadata file referencing the agent fingerprint.

For example, the encrypter 124 can hash the data 308 form a data identifier, which can represent the dataset to be used in deployment of the AI agent. As an example, the encrypter 124 may compute a SHA-256 digest for a customer data manifest represented by the data 308, which can yield a unique value linked to the corresponding data 308. The data identifier can be a hash of the manifest listing all customer-specific RAG documents or other local data sources. The data identifier can be used by as deployment system to mount the data 308. In some implementations, the encrypter 124 serializes the data 308 before hashing, which can maintain deterministic reproducibility of dataset references. For instance, canonicalization may involve key sorting or format normalization prior to digest computation to ensure consistency across environments.

The encrypter 124 can hash the configuration 312 to generate a configuration identifier, which can represent the customer- (or user) specific settings for deployment of the AI agent. For example, the configuration identifier can be consumed by the deployment system to apply environment variables, feature flags, or resource limits to the deployment of the AI agent. In some implementations, the configuration 312 may be encoded in a canonicalized JSON or YAML structure before processing by the encrypter 124. For instance, the structure can be flattened and normalized before hashing to prevent variations due to whitespace or system differences.

Referring further to FIG. 3, the system 300 can include a deployed fingerprint assembler 316. The deployed fingerprint assembler 316 can be analogous to or include components and/or functionality of the fingerprint assembler 128. The deployed fingerprint assembler 316 can generate a deployment metadata file that includes the agent fingerprint 132 and the hashed identifiers for user-specific components. The deployed fingerprint assembler 316 can perform data processing operations that merge these values into a canonical structure ready for encryption. In some implementations, the deployed fingerprint assembler 316 can construct a JSON object that defines fields such as for the agent fingerprint, the identifier of the data 308, and/or the identifier of the configuration 312. For example, the deployed fingerprint assembler 316 can retrieve the agent fingerprint 132, combine it with the computed identifiers from the encrypter 124, and assemble the combined fields into a structured object. In some implementations, the deployed fingerprint assembler 316 can apply canonicalization routines to establish consistent field ordering across computing environments. For example, the deployed fingerprint assembler 316 can alphabetically sort field names, normalize data types, and validate compliance with a predefined schema before passing the metadata to the encrypter 124 for hashing. The resulting canonical metadata file can be serialized using a deterministic encoding format so that identical logical content produces the same binary representation during subsequent encryption operations.

In some implementations, the deployed fingerprint assembler 316 generates the metadata file to include (one or more identifiers of) one or more deployment dependencies. For example, the identifier(s) can include an array of objects that can indicate an implementation of each corresponding dependency identifier declared in the agent fingerprint. The identifiers of the deployment dependencies can be used by the deployment orchestrator to configure network policies, firewall rules, or service mesh routes, for example and without limitation, such as to ensure that the AI agent can only communicate with its declared dependencies.

The following is an illustrative example of the metadata file generated by the deployed fingerprint assembler 316, including the identifiers and the schema for the metadata file:

{

“format_version”: “1.1”,

“manufacturer_id”: “87b1c428-2c67-428a-a195-21a4c4202c2e”,

“agent_model_name”: “MediBot-Nurse-v3.2-Intake”,

“timestamp”: “2025-08-27T10:00:00Z”,

“components”: {

“data_cid”:

“sha256: a1b2c3d4e5f678901234567890abcdef1234567890abcdef1234567890ab”,

“model_cid”: “sha256: b2c3d4e5f6a1234567890abcdef1234567890abcdef1234567890abcde”,

“instructions_cid”:

“sha256: c3d4e5f6a1b234567890abcdef1234567890abcdef1234567890abcd”,

“software_cid”: “sha256: d4e5f6a1b2c34567890abcdef1234567890abcdef1234567890abcde”

},

“external_dependencies_manifest”: [

{

“dependency_id”: “EMBEDDINGS_PROVIDER”,

“description”: “Service for generating text embeddings for RAG.”,

“interface_spec_cid”: “sha256: 2b3c4d5e6f7a8901234567890abcdef1234567890abcdef1234567890”

},

{

“dependency_id”: “PATIENT_LOOKUP_API”,

“description”: “Tool for retrieving patient records from an EMR.”,

“interface_spec_cid”: “sha256: 3c4d5e6f7a2b901234567890abcdef1234567890abcdef123456789”

}

]

}

Referring further to FIG. 3, the system 300 can use the encrypter 124 to generate a deployed agent fingerprint 320 based on the metadata file (e.g., the metadata file generated by the deployed fingerprint assembler 316). The deployed agent fingerprint 320 can represent a deterministic cryptographic identifier associated with a specific deployment instance of an AI agent that incorporates customer-specific components. In some implementations, the deployed agent fingerprint 320 can be generated by hashing, e.g., applying a cryptographic hash function such as SHA-256, to a canonicalized deployment metadata file representing the deployment-related parameters. For example, the system 300 can compute the fingerprint as SHA-256 (Canonicalize(DAF_Metadata_File)), thereby producing a reproducible identifier that establishes a verifiable association between the manufacturer definition and the customer deployment instance. In some implementations, the deployed agent fingerprint 320 can be transmitted to one or more validation systems, which can confirm component alignment and provenance across distinct customer environments. For example, a credential-granting authority can recompute a fingerprint directly from a deployed image and compare its value to the declared deployed agent fingerprint 320 to confirm that the deployment precisely matches the expected configuration. The deployed agent fingerprint 320 can be retained in a version-controlled repository or deployment register to facilitate subsequent verification or attestation of a specific release version. For example, the fingerprint can be stored as an immutable metadata tag linked to a corresponding container image of the AI agent in an artifact registry, establishing a persistent record of the deployed artifact for future reference.

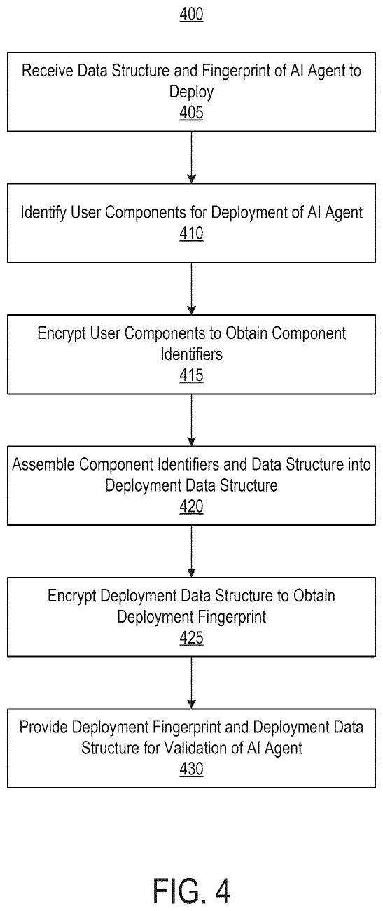

Referring now to FIG. 4, illustrated is a flowchart of a method 400 for generating and validating a deployment fingerprint for an AI agent. The method 400 can be executed, performed, or otherwise carried out by any of the computing systems described herein. In brief overview of the method 400, the method 400 can include receiving a data structure (e.g., metadata file) and fingerprint of AI agent to deploy 405, identifying user components for deployment of AI agent 410, encrypting user components to obtain component identifiers 415, assembling component identifiers and data structure into deployment data structure 420, encrypting deployment data structure to obtain a deployment fingerprint 425, and providing the deployment fingerprint and deployment data structure for validation of AI agent 430.

At 405, a data structure and a fingerprint of an AI agent to be deployed can be received. The data structure can include a metadata file, such as a metadata file defining a base configuration of the AI agent. In some implementations, the data structure and fingerprint can be obtained from a repository maintained by a manufacturer of record prior to deployment initialization. For example, retrieval can occur automatically through a continuous deployment pipeline, such as when a change to a deployment configuration is detected in a version control repository. The data structure and fingerprint can be accessed through secured interfaces, such as application programming interfaces or encrypted manifests, and verified for integrity through signed artifact exchange.

At 410, user-specific components required for deployment of the AI agent can be identified. The components can include customer data inputs and configuration files that define the deployment environment. In some implementations, the identification can occur after validation of the manufacturer's metadata file to confirm compatibility between manufacturer and customer-specific component definitions. For example, reference manifests can be inspected to locate resource files corresponding to domain-specific datasets and configuration templates. Identifiers, version descriptors, or asset paths defining each customer element can be determined to prepare for subsequent encryption of the user components.

At 415, encryption of the user-specific components can be performed to obtain component identifiers. Each deployment-specific manifest can be processed as an input to a cryptographic hashing operation. In some implementations, the user-specific components can be serialized and canonicalized before encryption to maintain deterministic output across environments. For example, manifest files can be normalized by ordering keys alphabetically and standardizing character encoding before computation of the hash values. The resulting identifiers can uniquely represent the data and configuration information used for deployment of the artificial intelligence agent.

At 420, the component identifiers and the received manufacturer data structure can be assembled into a deployment data structure. The deployment data structure can include combined fields, such as a base agent fingerprint, a customer data identifier, and a customer configuration identifier. In some implementations, the assembly process can occur automatically after all customer-specific hashing operations are completed. For example, a build pipeline may generate a JSON file once the component identifier values have been determined. Canonicalization steps can then be applied to ensure a deterministic representation, such as by sorting keys or aligning nested arrays according to schema specifications prior to serialization.

At 425, the deployment data structure can be encrypted to obtain a deployment fingerprint. The deployment fingerprint can result from a cryptographic hash computation that converts the canonicalized deployment data into a deterministic identifier. In some implementations, the fingerprint can be expressed as DAF=SHA-256 (Canonicalize(DAF_Metadata_File)). For example, cryptographic hashing can be performed during a final build stage of a deployment pipeline to produce an immutable release artifact. Whitespace removal and/or numeric normalization can be applied to the data structure before execution of the hashing procedure to maintain reproducibility of the output fingerprint across processing environments.

At 430, the deployment fingerprint and the associated deployment data structure can be provided for validation of the AI agent. The data can be made available to a credentialing or licensing entity for verification. In some implementations, the fingerprint and deployment data structure can be submitted as final artifacts within a deployment pipeline prior to instantiation of the agent within a compute cluster. For example, the data can be transmitted as signed objects through secure interfaces such as application programming interfaces to enable integrity comparison against a reference deployment fingerprint computed independently. Validation can include comparison of fingerprints to confirm alignment between the declared configuration and the deployed instance.

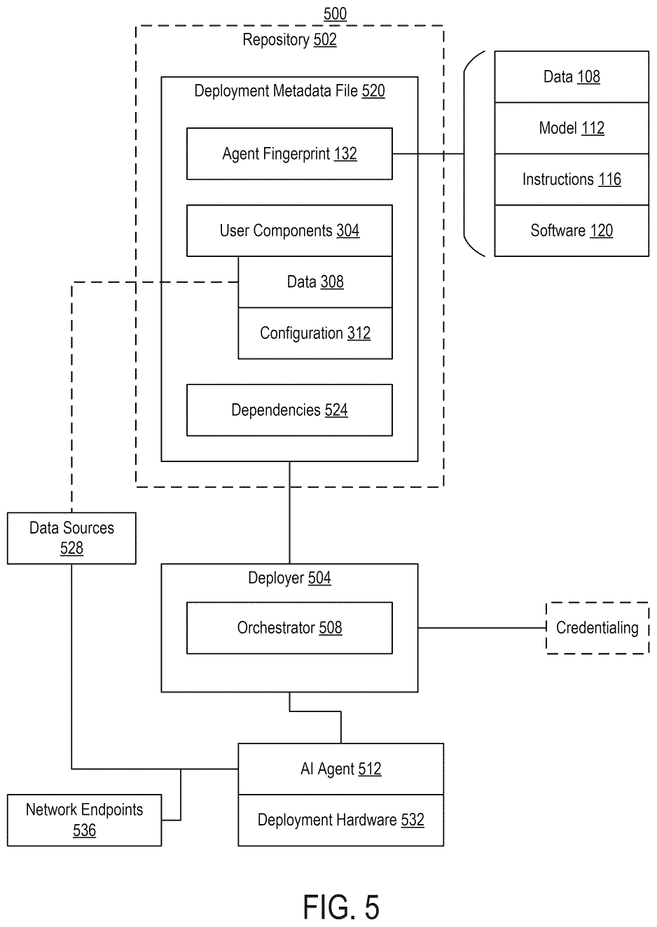

Referring now to FIG. 5, illustrated is a block diagram of an example of a system 500, such as an AI agent deployment system 500, in accordance with one or more implementations. The system 500 can use agent fingerprints, including deployed agent fingerprints, as both descriptive artifacts for auditing of AI agents as well as prescriptive blueprints for configuration-driven deployment, such as to allow the agent fingerprints to serve these dual purposes. The system 500 can be implemented by any of various entities described herein, including, for example, a customer or user of the AI agent, as well as a remote system that may be deploying the AI agent for credentialing or other verification-related purposes.

The system 500 can incorporate features of any of various systems described herein, such as the system 100 and the system 300. In brief overview, the system 500 can include a repository 502 for a deployment metadata file 520. The deployment metadata file 520 can include an agent fingerprint 132, user components 304 (including data 308 and configuration 312), and dependencies 524. The repository 502 can also reference base manufacturer components including data 108, model 112, instructions 116, and software 120. The system 500 can include or be coupled with one or more of a deployer 504 having an orchestrator 508, an AI agent 512, deployment hardware 532, data sources 528, and network endpoints 536.

Referring to FIG. 5 in further detail, the system 500 can include at least one deployer 504. The deployer 504 can include any one or more integrated or distributed computing systems, processors, hardware, software, firmware, functions, to perform operations such as deploying an AI agent, such as to deploy any of various agents described with respect to FIGS. 1-4 and/or AI agent 512. For example, the deployer 504 can be a computing system or service to instantiate the AI agent 512 based on a deployment metadata file. The system 500 can use the deployer 504 to execute a continuous integration and continuous delivery/deployment (CI/CD) process and/or a configuration-driven deployment system. For example, the deployer 504 may include a set of automated scripts or continuous delivery mechanisms that process fingerprints to launch corresponding workloads in a cloud environment. Such workloads may include tasks associated with or run for the AI agent, such as AI model training pipelines, inference services performing real-time predictions, or evaluation tasks executed across multiple containerized instances. In some implementations, these workloads may also be deployed within an on-premises environment, where the deployer 504 orchestrates local compute clusters or private data center nodes to provide similar fingerprint-based deployment and validation capabilities.

The deployer 504 can receive the deployment metadata file 520, and, as described further herein, can provision the components of the AI agent represented by the deployment metadata file 520, such as to deploy an instance of the AI agent 512. For example, the deployer 504 can generate provisioning operations for each component and/or configuration identified in the deployment metadata file 520. The deployer 504 can use one or more execution workflows to allocate compute resources for the instance of the AI agent 512, and can launch a runtime environment corresponding to the declared configuration. In some implementations, the deployer 504 can obtain container image digests and perform retrieval operations from a container registry to instantiate a software environment defined by the deployment metadata file 520. For example, the deployer 504 can use a container orchestration service to pull verified image digests, mount data assets, and apply runtime constraints that replicate the composition described in the metadata file 520. In some implementations, the deployer 504 can trigger initialization via the orchestrator 508, which interprets deployment manifests and executes runtime scheduling for the AI agent 512. For example, the deployer 504 can initiate a Google Kubernetes Engine (GKE) cluster or invoke Terraform workflows to allocate specific hardware resources, assign GPU node pools, and deploy the containerized agent workload in alignment with the specifications recorded in the deployment metadata file 520.

Referring further to FIG. 5, the deployer 504 can include or be coupled with an orchestrator 508. The orchestrator 508 can orchestrate (e.g., coordinate and/or control) automated deployment and/or runtime execution of the AI agent 512. The orchestrator 508 can coordinate deployment of the instance of the AI agent 512 based on the deployment metadata file 520, such as to generate and/or executing provisioning operations to instantiate the AI agent 512 according to one or more components and/or identifiers in the deployment metadata file 520. The orchestrator 508 can interpret the deployment metadata file 520 as a prescriptive manifest that defines parameters for container instantiation, software image retrieval, and/or resource provisioning. In some implementations, the orchestrator 508 can initiate a sequence of provisioning tasks that assign compute nodes, allocate storage volumes, or configure networking interfaces according to specifications defined by the metadata file 520. For example, the orchestrator 508 can request container image digests from a registry, can download model artifacts, and can attach persistent data volumes corresponding to the data 308 field in the metadata file 520. The orchestrator 508 can schedule containers based on declared resource constraints, such as GPU type, memory size, or processor limits, and can apply runtime configuration variables defined in customer-specific entries of the metadata file 520.

In some implementations, the orchestrator 508 can enforce deployment policies (e.g., according to dependencies 524) to maintain operational fidelity between instantiated workloads and declared dependencies. The orchestrator 508 can map bindings defined in dependency entries to concrete service endpoints and can configure the runtime environment to restrict external traffic accordingly. For example, the orchestrator 508 can generate Kubernetes network policy manifests that confine egress connectivity to APIs or external services explicitly identified in dependencies 524. The orchestrator 508 can delay model service initialization until preloaded data assets from data sources 528 are mounted, ensuring deterministic correspondence between the deployed configuration and the structural definition recorded in the deployment metadata file 520.

Referring further to FIG. 5, the system 500 can include or operate at least one AI agent 512. The AI agent 512 can include any one or more of the AI agents described with reference to FIGS. 1-4, including agents associated with an agent fingerprint 132 or a deployed agent fingerprint 320. The AI agent 512 can correspond to a deployed instance that the deployer 504 instantiates according to the deployment metadata file 520. In some implementations, the AI agent 512 can execute functions defined by the component identifiers, configuration parameters, and dependency bindings within the deployment metadata file 520 to reproduce the agentic behavior specified by the manufacturer and extended by customer-specific elements.

As depicted in FIG. 5, the system 500 can include or be coupled with a repository 502. The repository 502 can include any of various storage systems, such as any of various local, on-premises, or cloud-based systems for storing data including agent fingerprints and metadata files such as the deployment metadata file 520. In some implementations, the repository 502 can include distributed object storage, relational databases, or document-oriented storage platforms that provide version-controlled access to build artifacts. For example, the repository 502 can be implemented using cloud-based services that store metadata objects and container digests in association with unique identifiers representing release versions of the AI agent. In some implementations, the repository 502 can include local or hybrid systems that provide fast access to component manifests used during continuous integration and deployment operations. For example, the repository 502 can include an on-premises artifact registry that stores container images for the software 120 and corresponding metadata structures required to instantiate a verified deployment of the AI agent. The repository 502 can operate as a version-controlled storage system that stores metadata files, component manifests, and configuration definitions used for AI agent deployment. In some implementations, the repository 502 can maintain different versions of deployment metadata within a Git-based repository or a document-oriented database. For example, each version of a deployment file can correspond to a specific commit identifier or timestamp associated with an update operation. In some implementations, the deployer 504 can monitor the deployment metadata file 520 (e.g., as stored in the repository 502), and can trigger one or more operations based on detection of a change to the deployment metadata file 520, such as to retrieve the deployment metadata file 520, or re-validate or re-deploy the AI agent 512.

Referring further to FIG. 5, the system 500 can store or receive one or more deployment metadata files 520, such as to obtain the deployment metadata files 520 from the repository 502. The deployment metadata file 520 can correspond to any of various deployment metadata files described with reference to FIGS. 1-4. The deployment metadata files 520 can be JSON files, for example and without limitation. In some implementations, the deployment metadata file 520 can be used by the orchestrator 508 as a prescriptive blueprint for deploying the AI agent 512 on deployment hardware 532. For example, the orchestrator 508 can parse the deployment metadata file 520 to extract identifiers (e.g., hashes) of components for the AI agent 512, such as to retrieve container image digests associated with the software 120 and model 112 specified in the base agent fingerprint 132, and can execute retrieval operations to load those components into the deployment environment. The orchestrator 508 can interpret dependency bindings within the deployment metadata file 520 to allocate network endpoints 536 and data sources 528 and to establish the declared service connectivity prior to runtime activation of the AI agent 512. In some implementations, deterministic hash references within the deployment metadata file 520 can enable independent verification that the components loaded by the orchestrator 508 correspond exactly to those declared in the metadata structure and intended by the manufacturer of record.

The deployment metadata file 520 can include one or more configuration parameters and/or identifiers that define the instance of the AI agent 512, such as to allow the deployer 504 to use the deployment metadata file 520 as a prescriptive blueprint for the instance of the AI agent 512. In some implementations, the deployment metadata file 520 can include the agent fingerprint 132 (e.g., a base agent fingerprint 132), which can be used to identify the data 108, model 112, instructions 116, and/or software 120 (e.g., as described with reference to FIG. 1). The deployment metadata file 520 can include the (identifier of) the user component(s) 304, and can include the (identifier of) the configuration 312, which can allow the deployer 504 to deploy the AI agent 512 according to such user-specific data and functionality. As described above with respect to FIGS. 1-4, the identifiers in the deployment metadata file 520 can be used as pointers to the corresponding components, which the deployer 504 can extract (e.g., as hashes) to obtain the corresponding components.

As depicted in FIG. 5, the deployment metadata file 520 can include one or more identifiers of one or more dependencies 524. The dependencies 524 can indicate one or more electronic and/or networked resources, services, or interfaces for components useful for execution of the AI agent 512, such as tokenizers or embedding functions. For example, the dependencies 524 can define the external service interfaces and/or operational bindings recorded within the deployment metadata file 520, including but not limited to the dependencies in the example of the deployment metadata file described with reference to FIG. 3, the deployed fingerprint assembler 316, and the deployed agent fingerprint 320. Each dependency entry can specify a relationship between a declared dependency identifier and the corresponding runtime endpoint that the AI agent 512 may access during execution. In some implementations, the dependencies 524 can enumerate machine-readable specifications identifying services such as external application programming interfaces (APIs), structured data repositories, or third-party runtime connectors, among others. For example, the dependencies 524 can include explicit records describing an embeddings service, a patient record retrieval system, or a cloud-based analytics endpoint, each referenced by a respective dependency identifier and stored universal resource identifier (URI) field. Each dependency entry can include one or more parameters that identify the service name, URI, version identifier, or protocol standard defining how the AI agent 512 communicates with the associated resource. The dependencies 524 can provide a deterministic mapping between the configuration of the AI agent 512 and the specific network entry points required for deployment of the AI agent 512.

In some implementations, the orchestrator 508 can process the dependencies 524 within the deployment metadata file 520 to generate one or more network policies. The deployer 504 can use the network policies to control data to be received by and/or transmitted from the AI agent 512. For example, the deployer 504 can control runtime egress behavior of the deployed instance of the AI agent 512 according to the one or more network policies. For example, the orchestrator 508 can construct and apply policy manifests (e.g., Kubernetes network policies) that permit outbound communications exclusively to URIs declared within the dependency entries of the metadata file 520. The dependencies 524 can define one or more authentication or protocol attributes, such as access token policies or mutual transport layer security (mTLS) requirements, that the orchestrator 508 may apply to network interface configurations.

As depicted in FIG. 5, the deployer 504 can use the identifiers of data 308 to receive data from one or more data sources 528. The data sources 528 can include one or more external databases, document repositories, and/or application programming interfaces that the AI agent 512 accesses during operation to obtain real-time or static information correlated to one or more tasks executed by the AI agent 512. In some implementations, the data sources 528 can include interfaces to structured or unstructured storage systems maintained by a customer, such as enterprise resource records, analytical datasets, or contextual resource catalogs. For example, the data sources 528 can include healthcare record servers, inventory management databases, or telemetry data feeds associated with the customer's environment. The data sources 528 can provide data objects or structured outputs that the deployer 504 or orchestrator 508 can retrieve based on the dependencies 524. In some implementations, the data sources 528 can deliver information directly to the AI agent 512 through request-response transactions declared in the deployment metadata file 520. For example, in a medical intake system, the data sources 528 can supply patient identifiers, historical encounter data, or form templates that the AI agent 512 incorporates into dialogue generation and task completion. The data sources 528 can communicate using secure transport connections and authentication methods explicitly specified by the dependency declarations in the deployment metadata file 520, such as OAuth2 or mutual transport layer security authentication protocols, among others. For example, OAuth2 or mutual TLS tokens specified in the dependency fields may be applied when establishing network connections to external data services.

In some implementations, the deployer 504 deploys the AI agent 512 on deployment hardware 532, such as deployment hardware 532 identified in the deployment metadata file 520. The system 500 can include the deployment hardware 532 or can be communicatively coupled with the deployment hardware 532. The deployment hardware 532 can include the physical or virtual compute infrastructure on which the AI agent 512 operates. In some implementations, the deployment hardware 532 can include multiple processing nodes or virtual machines provisioned by the orchestrator 508 to execute workloads that represent the instantiated AI agent 512. For example, the deployment hardware 532 can include GPU-accelerated instances, such as NVIDIA A100 or H100 devices, or CPU-based compute nodes that execute containerized environments for software 120 identified in the deployment metadata file 520. In some implementations, the deployer 504 can cause the deployment hardware 532 can retrieve data according to the deployment metadata file 520, such as to retrieve one or more container images, model checkpoints, and configuration files identified by corresponding component identifiers, which the deployer 504 can use to construct the runtime environment for inference or training operations. For example, the orchestrator 508 can allocate resources matching the specifications declared in the deployment metadata file 520, such as GPU type, driver version, or memory capacity, and can attach persistent volumes that store the model 112 or data 108 to the designated compute instance. The deployment hardware 532 can execute initialization sequences to load neural network weights, establish memory mappings for tensor operations, and apply container runtime policies that define the compute scope of the AI agent 512 during operation.

The system 500 can be coupled with one or more network endpoints 536. The deployer 504 can connect the instance of the AI agent 512 to the one or more network endpoints 536 according to the dependencies 524. For example, the dependencies 524 can be identifiers of the one or more network endpoints 536. The network endpoints 536 can represent remote addresses or uniform resource identifiers declared in the dependency bindings recorded within the deployment metadata file. In some implementations, the network endpoints 536 can identify one or more application programming interface domains or other remote services to which the AI agent 512 is permitted to transmit requests during execution. For example, the network endpoints 536 can include addresses corresponding to a text embedding service, a document retrieval application programming interface, or a third-party analytics service, among others. The deployer 504 can use the network endpoints 536 to apply network access restrictions that limit traffic within a deployed environment to those specified destinations. In some implementations, the orchestrator 508 can generate a network policy specifying egress routes that correspond exclusively to the declared network endpoints 536 contained in the deployment metadata file. For example, the orchestrator 508 can generate policy definitions that constrain outbound connections to the set of uniform resource identifiers included in the dependency bindings, thereby aligning the deployed communication topology with the configuration declared by the manufacturer of record. The network endpoints 536 can further be used as reference parameters for validating declared dependencies during credential evaluation, such as by verifying that external service calls conform to the addresses and protocols enumerated in the dependency bindings of the deployment metadata file.

For example, the deployer 504 parse the deployment metadata file 520 to identify the component identifiers in the deployment metadata file 520, and can provision the identified components, such as the data 108, model 112, instructions 116, and software 120, along with the data 308 and/or configuration 312, to deploy the instance of the AI agent 512 on the deployment hardware 532. For example, the deployer 504 can use the orchestrator 508 to pull versioned container images, load configuration parameters, and attach runtime resources so that an instance of the AI agent 512 executes according to the deployment metadata file 520. In some implementations, the deployer 504 establishes connections between the orchestration hardware 532 and at least one of the data sources 528 or the network endpoints 536 to allow the AI agent 512 to access external resources in accordance with the deployment metadata file 520, such as to access task-specific resources for any one or more tasks that the AI agent 512 is to perform.

In some implementations, the deployer 504 validates the deployment metadata file 520 against one or more fingerprints, e.g., against at least one of the agent fingerprint 132 (e.g., as provided by the manufacturer of record) or the deployed agent fingerprint 320 (e.g., as provided by a customer or user). The deployer 504 can compute a hash of each component of the plurality of components identified in the deployment metadata file 520 (e.g., as retrieved from the repository 502). In some implementations, each component of the deployment metadata file 520, such as the data 108, model 112, instructions 116, software 120, user data 308, or configuration 312, can be serialized and processed through a cryptographic hash function, generating deterministic component identifiers used for validation. The deployer 504 can maintain a same canonical serialization structure as to be applied during building of the AI agent 512, which can allow for equivalent input manifests to yield identical output hashes.

The deployer 504 can assemble a second metadata file based on the hash of each component and a structure of the deployment metadata file 520. In some implementations, the deployer 504 can use the orchestrator 508 to integrate the computed hashes into a canonicalized metadata object formatted according to the same schema defined by the manufacturer of record. For example, the deployer 504 can write a composite object that includes the base agent fingerprint 132, all derived component identifiers, and any dependency bindings 524 declared for the runtime environment. The second metadata file can preserve the hierarchical arrangement of the deployment metadata file 520 so that downstream verification systems can recompute the agent fingerprint without ambiguity.

The deployer 504 can validate the deployment metadata file 520 based on a hash of the second metadata file matching at least one of the agent fingerprint 132 or the deployed agent fingerprint 320. The validation operation can be executed by the orchestrator 508 under the direction of the deployer 504. In some implementations, the orchestrator 508 can perform a verification hash on the assembled metadata object and compare the resulting value to the agent fingerprint 132 received from the repository 502. For example, if the computed hash matches the stored fingerprint, the deployer 504 can determine that the deployment metadata file 520 represents a verified configuration corresponding to the defined version of the AI agent 512. In some implementations, the deployer 504 can condition subsequent provisioning of the AI agent 512 on successful completion of this validation process, thereby linking runtime instantiation on deployment hardware 532 to a deterministically verified configuration state defined by the canonical fingerprint. For example, the deployer 504 can provision the components for the AI agent 512, as indicated in the deployment metadata file 520, responsive to the validation of the deployment metadata file 520.

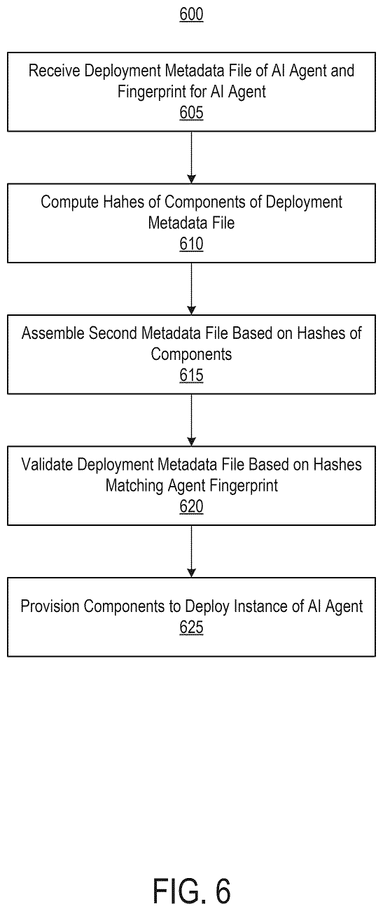

Referring now to FIG. 6, illustrated is a flow chart of a method 600, such as a method for deploying an AI agent according to a configuration-driven deployment process. The method 600 can be executed, performed, or otherwise carried out by any of the computing systems or devices described herein. The method 600 or one or more operations of the method 600 can be triggered, for example, by a request to deploy, update, or verify an AI agent, or responsive to detection of a change in one or more components of the AI agent, including but not limited to based on detection of a change to an agent fingerprint or a deployed agent fingerprint. In brief overview of the method 600, the method 600 can include receiving a deployment metadata file and an agent fingerprint 605, computing hashes of components of the deployment metadata file 610, assembling a second metadata file based on the hashes of components 615, validating the deployment metadata file based on the fingerprint 620, and provisioning components to deploy an instance of the AI agent 625.

At 605, the method 600 can include receiving a deployment metadata file and an agent fingerprint. The deployment metadata file can specify structural parameters and component identifiers to instantiate an instance of an AI agent. The agent fingerprint can provide a deterministic identifier of a configuration of the AI agent, such as base configuration (e.g., a base configuration defined by a first entity, such as a manufacturer, where one or more downstream customers or users may specify additional configuration for building on top of the base configuration). In some implementations, a deployment orchestrator can detect a repository change and automatically retrieve the corresponding metadata file and fingerprint from a version-controlled repository or artifact registry. For example, when a manufacturer releases a new agent build, the orchestrator can receive the associated deployment metadata file and fingerprint from an encrypted storage system registered to the manufacturer, ensuring the input artifacts correspond to a valid release version.

At 610, the method 600 can include computing hashes of the components identified in the deployment metadata file. The computation can be performed by applying a cryptographic operation on each component, such as to apply SHA-256 to the identifiers and/or manifests of each component. In some implementations, hash inputs can include customer-specific datasets, configuration definitions, or declared service dependencies represented within the deployment metadata file.

At 615, the method 600 can include assembling a second metadata file based on the component hashes. For example, the hash outputs corresponding to each component can be aggregated into a canonicalized data structure. In some implementations, the assembler can mirror the schema of the original deployment metadata file while substituting component references with their respective hash values, forming a derivative object defining a deterministic view of the deployment composition. The resulting second metadata file can represent a normalized form suitable for comparison against the received agent fingerprint.

At 620, the method 600 can include validating the deployment metadata file based on the fingerprint. The validation can be performed based on computing a hash of the second metadata file, and comparing the hash of the second metadata file to the agent fingerprint. For example, the comparison can be performed to detect any differences between the hashes, such as to verify integrity and consistency between declared and actual configurations. In some implementations, validation can act as a gating process prior to deployer initialization. For example, if the hash of the second metadata file matches the fingerprint, the metadata file can be marked as consistent with the authenticated agent definition. The validation process can thereby confirm that all component identifiers, dependencies, and structural parameters correspond to the agent version intended for deployment. The confirmation of equivalence can enable further automated actions such as provisioning of resources for the identified configuration.

At 625, the method 600 can include provisioning components to deploy an instance of the AI agent. The provisioning can be performed by instantiating an operational environment specified by the validated metadata file. In some implementations, provisioning can include retrieving software containers, model parameters, datasets, and/or configuration files corresponding to the component identifiers confirmed during validation. For example, one or more compute nodes can be allocated within selected deployment hardware, including, for example and without limitation, one or more CPU or GPU nodes, to load verified container images representing the software and model referenced in the deployment metadata file. Data indicated in the deployment metadata file can be mounted, and network policies associated with dependency entries specifying authorized endpoints can be implemented. The deployment process can produce an instantiated AI agent, which can be run with deterministic fidelity to the fingerprinted configuration described within the validated metadata structure.

Systems and methods as described herein can be implemented by any of various neural networks and/or machine learning models. These can include, for example and without limitation, one or more neural networks (or layers, nodes, weights, and/or biases thereof), convolutional neural networks, recurrent neural networks, attention networks, transformer networks, encoders, decoders, sequence to sequence models, generative models, pretrained models, diffusion models, multimodal models, generative adversarial networks, or various combinations thereof, which may be configured (e.g., trained, fine-tuned, having transfer learning performed, updated or operated by in-context learning, examples, or prompting, etc.) through operations such as supervised learning, self-supervised learning, or unsupervised learning. Systems and methods as described herein can be implemented in any of various artificial intelligence architectures or processing pipelines, including, for example, agentic pipelines, retrieval-based pipelines (e.g., retrieval-augmented generation), or various combinations thereof.

Having now described some illustrative implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed in connection with one implementation are not intended to be excluded from a similar role in other implementations or implementations.

The hardware and data processing components used to implement the various processes, operations, illustrative logics, logical blocks, modules and circuits described in connection with the implementations disclosed herein can be implemented or performed with a general purpose single-or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor can be a microprocessor, or, any conventional processor, controller, microcontroller, soc (system on chip), som (system on module) or state machine. A processor also can be implemented as a combination of computing devices, such as a combination of a DSP and a microprocessor, a plurality of microprocessors, one or more microprocessors in conjunction with a DSP core, or any other such configuration. In some implementations, particular processes and methods can be performed by circuitry that is specific to a given function. The memory (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present disclosure. The memory can be or include volatile memory or non-volatile memory, and can include database components, object code components, script components, or any other type of information structure for supporting the various activities and information structures described in the present disclosure. According to an exemplary implementation, the memory is communicably connected to the processor via a processing circuit and includes computer code for executing (e.g., by the processing circuit and/or the processor) the one or more processes described herein.

The present disclosure contemplates methods, systems and program products on any machine-readable media for accomplishing various operations. The implementations of the present disclosure can be implemented using existing computer processors, or by a special purpose computer processor for an appropriate system, incorporated for this or another purpose, or by a hardwired system. Implementations within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation or implementation, and references to “an implementation,” “some implementations,” “one implementation” or the like are not necessarily mutually exclusive and are intended to indicate that a particular feature, structure, or characteristic described in connection with the implementation can be included in at least one implementation or implementation. Such terms as used herein are not necessarily all referring to the same implementation. Any implementation can be combined with any other implementation, inclusively or exclusively, in any manner consistent with the aspects and implementations disclosed herein.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Systems and methods described herein can be embodied in other specific forms without departing from the characteristics thereof. Further relative parallel, perpendicular, vertical or other positioning or orientation descriptions include variations within +/−10% or +/−10 degrees of pure vertical, parallel or perpendicular positioning. References to “approximately,” “about” “substantially” or other terms of degree include variations of +/−10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

The term “coupled” and variations thereof includes the joining of two members directly or indirectly to one another. Such joining can be stationary (e.g., permanent or fixed) or moveable (e.g., removable or releasable). Such joining can be achieved with the two members coupled directly with or to each other, with the two members coupled with each other using a separate intervening member and any additional intermediate members coupled with one another, or with the two members coupled with each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling can be mechanical, electrical, or fluidic.

References to “or” can be construed as inclusive so that any terms described using “or” can indicate any of a single, more than one, and all of the described terms. A reference to “at least one of ‘A’ and ‘B’” can include only ‘A’, only ‘B’, as well as both ‘A’ and ‘B’. Such references used in conjunction with “comprising” or other open terminology can include additional items.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

References herein to the positions of elements (e.g., “top,” “bottom,” “above,” “below”) are merely used to describe the orientation of various elements in the FIGURES. The orientation of various elements can differ according to other exemplary implementations, and that such variations are intended to be encompassed by the present disclosure.

What is claimed is:

1. A system, comprising:
one or more processors to:
receive a deployment metadata file of an AI agent and a fingerprint for the AI agent, the deployment metadata file comprising a plurality of identifiers of a plurality of components for deployment of an instance of the AI agent;
compute a hash of each component of the plurality of components identified in the deployment metadata file and retrieved from storage;
assemble a second metadata file based on the hash of each component of the plurality of components and a structure of the deployment metadata file;
validate the deployment metadata file based on a hash of the second metadata file matching the fingerprint for the AI agent; and
responsive to validating the deployment metadata file, provision each of the plurality of components identified in the deployment metadata file to deploy the instance of the AI agent.

2. The system of claim 1, wherein the one or more processors are to trigger at least the reception of the deployment metadata file based on detection of a change to the deployment metadata file in a repository.

3. The system of claim 1, wherein the one or more processors are to:
parse the deployment metadata file to extract one or more hashes representing an identifier of at least one of a software component for deployment of the AI agent, a configuration for deployment of the AI agent, or a dependency indicating a networking policy for the AI agent; and
deploy the AI agent according to the one or more hashes.

4. The system of claim 1, wherein the one or more processors are to retrieve the plurality of components from a storage system provided by a manufacturer of the AI agent.

5. The system of claim 1, wherein the one or more processors are to load instructions for deployment of the AI agent at runtime according to an identifier of the instructions in the plurality of identifiers.

6. The system of claim 1, wherein the fingerprint of the AI agent comprises a hash of a metadata file of the AI agent.

7. The system of claim 1, wherein the one or more processors are to output the deployment metadata file for credentialing the deployment of the AI agent.

8. The system of claim 1, wherein the plurality of components including at least one of user-specific data for deployment of the AI agent or user-specific configuration information for configuration of the AI agent.

9. The system of claim 1, wherein the plurality of identifiers include at least one identifier for a hardware resource on which to deploy the AI agent.

10. The system of claim 1, wherein the plurality of identifiers indicate one or more dependencies for the deployment of the AI agent.

11. A method of AI agent deployment, comprising:
receiving, by one or more processors, a deployment metadata file of an AI agent and a fingerprint for the AI agent, the deployment metadata file comprising a plurality of identifiers of a plurality of components for deployment of an instance of the AI agent;
computing, by the one or more processors, a hash of each component of the plurality of components identified in the deployment metadata file and retrieved from storage;
assembling, by one or more processors, a second metadata file based on the hash of each component of the plurality of components and a structure of the deployment metadata file;
validating, by one or more processors, the deployment metadata file based on a hash of the second metadata file matching the fingerprint for the AI agent; and
responsive to validating the deployment metadata file, provisioning each of the plurality of components identified in the deployment metadata file to deploy the instance of the AI agent.

12. The method of claim 11, comprising triggering at least the reception of the deployment metadata file based on detection of a change to the deployment metadata file in a repository.

13. The method of claim 11, comprising:
parsing the deployment metadata file to extract one or more hashes representing an identifier of at least one of a software component for deployment of the AI agent, a configuration for deployment of the AI agent, or a dependency indicating a networking policy for the AI agent; and
deploying the AI agent according to the one or more hashes.

14. The method of claim 11, comprising retrieving the plurality of components from a storage system provided by a manufacturer of the AI agent.

15. The method of claim 11, comprising loading instructions for deployment of the AI agent at runtime according to an identifier of the instructions in the plurality of identifiers.

16. The method of claim 11, wherein the fingerprint of the AI agent comprises a hash of a metadata file of the AI agent.

17. The method of claim 11, comprising outputting the deployment metadata file for credentialing the deployment of the AI agent.

18. The method of claim 11, wherein the plurality of components including at least one of user-specific data for deployment of the AI agent or user-specific configuration information for configuration of the AI agent.

19. The method of claim 11, comprising deploying the AI agent on one or more hardware wherein the plurality of identifiers include at least one identifier for a hardware resource on which to deploy the AI agent.

20. The method of claim 11, comprising detecting, using the plurality of identifiers, one or more dependencies for the deployment of the AI agent.
